(19) United States
(12) Patent Application Publication
(10) Pub. No.: US 2021/0198654 A1
(43) Pub. Date: Jul. 1, 2021

(54) METHOD FOR ISOLATION AND HARVESTING MICROVESICLES

(71) Applicant: Jerome Canady Research Institute for Advanced Biological and Technological Sciences, Takoma Park, MD (US)

(72) Inventors: Jerome Canady, Lakeland, FL (US); Saravana Murthy, Owings Mills, MD (US); Xiaoqian Cheng, Fairfax, VA (US); Taisen Zhuang, Rockville, MD (US)

(21) Appl. No.: 17/135,847

(22) Filed: Dec. 28, 2020

Related U.S. Application Data
(60) Provisional application No. 62/953,801, filed on Dec. 26, 2019.

(51) Int. Cl. C12N 13/00; C12N 5/0783

(52) U.S. Cl. CPC C12N 13/00; C12N 2529/00; C12N 5/0636

(57) ABSTRACT

A method for isolation and culturing of T Lymphocytes from whole blood and harvesting exosomes. The method comprises isolating peripheral blood mononuclear cells from human blood, culturing the isolated peripheral blood mononuclear cells, treating the cultured peripheral blood mononuclear cells with cold atmospheric plasma for secretion of microvesicles, and harvesting microvesicles from the CAP-treated peripheral blood mononuclear cells.

### CROSS-REFERENCE TO RELATED APPLICATIONS

The present application claims the benefit of the filing date of U.S. Provisional Patent Application Ser. No. 62/953,801 filed by the present inventors on Dec. 26, 2019.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to bioengineering exosomes for cancer therapy utilizing cold atmospheric plasma.

### Brief Description of the Related Art

There is exciting potential for exosomes as therapeutic vehicles for cancer treatment. Successful implementation in the clinical setting will be dependent upon establishment of rigorous standards for exosome manipulation, isolation, and characterization. See, K. Gilligan and R. Dwyer, “Engineering Exosomes for Cancer Therapy,” Int. J. Mol. Sci. 2017, 18, 1122. See also International Patent Application WO 2017/049166 entitled “CAR T CELL THERAPIES WITH ENHANCED EFFICACY.”

Exosomes are small extracellular vesicles with diameters of 30-150 nm. In both physiological and pathological conditions, nearly all types of cells can release exosomes, which play important roles in cell communication and epigenetic regulation by transporting crucial protein and genetic materials such as miRNA, mRNA, and DNA. Historically, the two main technical hindrances that have restricted the basic and applied research of exosomes include, first, how to simplify the extraction and improve the yield of exosomes and, second, how to effectively distinguish exosomes from other extracellular vesicles, especially functional microvesicles. See, Fais S, O'Driscoll L, Borras F E, Buzas E, Camussi G, Cappello F, et al. Evidence-based clinical use of nanoscale extracellular vesicles in nanomedicine. Acs Nano. 2016; 10: 3886-99; Karimi N, Cvjetkovic A, Jang S C, Crescitelli R, Feizi M A H, Nieuwland R, et al., “Detailed analysis of the plasma extracellular vesicle proteome after separation from lipoproteins. Cell Mol Life Sci. 2018; 75: 2873-86.”

Over the past few decades, although a standardized exosome isolation method has still not become available, a number of techniques have been established through exploration of the biochemical and physicochemical features of exosomes. A summary of such techniques is provided in D. Yang, et al., “Progress, opportunity, and perspective on exosome isolation—efforts for efficient exosome-based theranotics,” Theranostics, 2020; 10(8) 3684-3707.

Recently immunotherapy such as chimeric antigen receptors (CARs) has brought new paradigm in cancer immunotherapy, wherein a patient's own T cells are bioengineered to express CARs that identify, attach to, and subsequently kill tumor cells. Moreover, checkpoint blockade, adoptive cell transfer, human recombinant cytokines and cancer vaccines have shown very encouraging signs for cancer treatment, however only a subset of patients show complete response to these treatments. The principle of cancer immunotherapy is based on the identification of tumor-associated antigens (TAAs) which are dysregulated mutated gene products that are presented as antigens and neutralization of these cells by engineered T cells. However, the sparse expression of these antigens and loss of neoantigen during malignancy are insufficient to prompt a full-blown immune response to neutralize the tumor. Moreover, these therapies have other limitations that directly affect patients, some of these are cytokine release syndrome (CRS) and CAR T-related encephalopathy syndrome (CRES), long vein-to-vein time, treatment is restricted to heavily pretreated patients, multistep process of generating autologous CAR T cells increases the risk of production failure and commercial scalability challenges. The foregoing references are hereby incorporated by reference in their entirety.

### SUMMARY OF THE INVENTION

The objective of the present invention is isolation and harvesting microvesicles or apoptotic bodies such as T lymphocyte exosomes realized from Glioblastoma Cell line U87. For this, cold atmospheric plasma treatment was performed on U87 cells and the secreted exosomes were harvested by polymer precipitation-based method. These exosomes were applied to CAP untreated U87 cells. After 24 hours of exosome treatment the U87 cells showed significant increase in apoptosis and cell death in imaging and flow cytometry analysis. These preliminary results suggest that exosomes realized from the cancer cells after CAP treatment is a potent inducer of cell death. The mechanistic study of exosomes realize after CAP treatment and complete analysis of exosomes cargo is warranted.

In a preferred embodiment, the present invention is a method for isolation and culturing of microvesicles. The method comprises isolating peripheral blood mononuclear cells from human blood, culturing the isolated peripheral blood mononuclear cells, treating the cultured peripheral blood mononuclear cells with cold atmospheric plasma for secretion of microvesicles, and harvesting microvesicles from the CAP-treated peripheral blood mononuclear cells. The harvested microvesicles comprise T lymphocyte exosomes. The step of isolating peripheral blood mononuclear cells from human blood may comprise adding human blood to a polymorph density medium in a first flask, centrifuging the first flask of blood and polymorph density medium to separate the peripheral blood mononuclear cells from other blood components, collecting the peripheral blood mononuclear cells from the first flask and transferring the peripheral blood mononuclear cells to a second flask, centrifuging the second flask of peripheral blood mononuclear cells, and washing the peripheral blood mononuclear cells in the second flask. The step of culturing the peripheral blood mononuclear cells may comprise culturing the washed peripheral blood mononuclear cells in selective media, removing the peripheral blood mononuclear cells from the selective media and transferring them to a third flask, centrifuging the peripheral blood mononuclear cells in the third flask, transferring the centrifuged peripheral blood mononuclear cells from the third flask to a fourth flask, centrifuging the peripheral blood mononuclear cells in the fourth flask, culturing the centrifuged peripheral blood mononuclear cells in the fourth flask in selective media containing IL-2 or IL-15, and replacing the selective media with exosome free fetal bovine serum. The step of harvesting microvesicles from the CAP-treated peripheral blood mononuclear cells comprises an isolation method. The isolation method may comprise incubating the CAP-treated peripheral blood mononuclear cells, differentially centrifuging the incubated CAP-treated peripheral blood mononuclear cells, mixing the centrifuged incubated CAP-treated peripheral blood mononuclear cells with an exosome isolation solution, incubating the exosome isolation solution and centrifuged incubated CAP-treated peripheral blood mononuclear cells, centrifuging the incubated exosome isolation solution and centrifuged incubated CAP-treated peripheral blood mononuclear cells, and collecting microvesicle pellets from the centrifuged incubated exosome isolation solution and centrifuged incubated CAP-treated peripheral blood mononuclear cells.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a flow chart of a protocol for isolation and culturing Of T Lymphocytes from whole blood and harvesting exosomes in accordance with a preferred embodiment of the present invention.

### DETAILED DESCRIPTION OF THE PREFERRED EMBODIMENTS

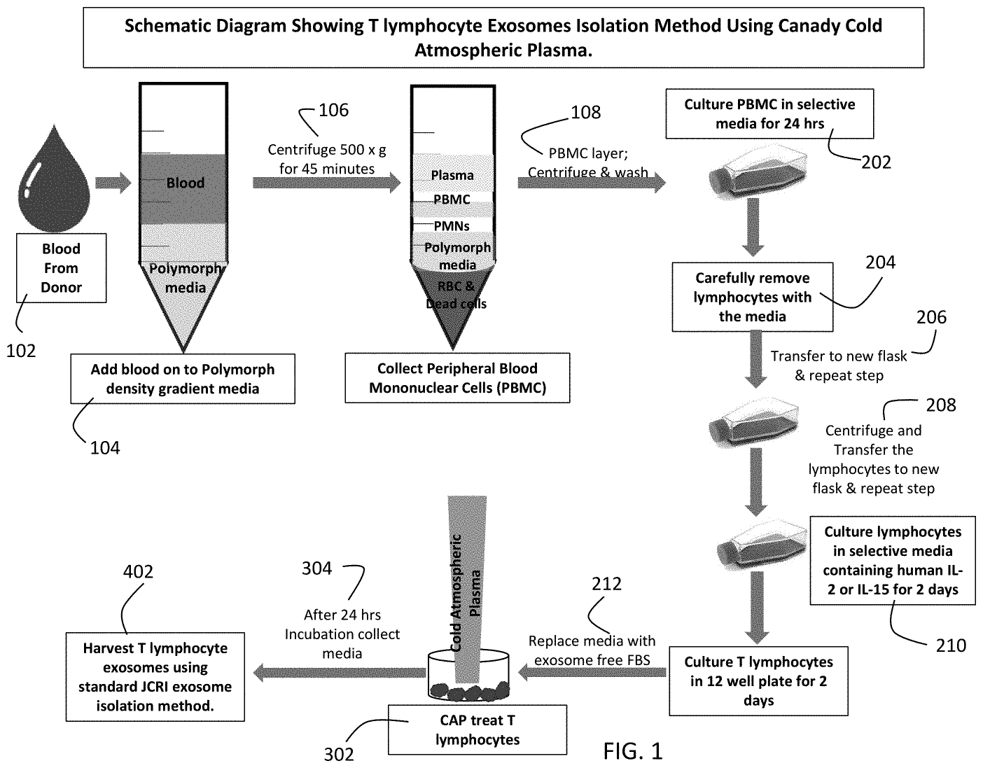

The present invention is described with reference to the drawing. A preferred embodiment of the present invention for isolating and harvesting microvesicles is described with reference to FIG. 1. Human blood obtained from a donor (102) is added to a polymorph density medium (104). The blood and polymorph solution is then centrifuged (106), for 45 minutes in the example shown in FIG. 1. The peripheral blood mononuclear cells (PBMC), which now will have separated from the other blood components, are removed, centrifuged and washed (108). The PMBC is then cultured in a selective media for 24 hours (202). The lymphocytes are then removed with the media (204) and transferred to a new flask and the centrifuge step is repeated (206). The lymphocytes are then centrifuged, transferred to a new flask, and the step if repeated (208). The lymphocytes are then cultured in selective media containing IL-2 or IL-15 for 2 days (210). The media is then replaced with exosome free FBS (212). The T lymphocytes are then treated with cold atmospheric plasma (302). Following CAP treatment, the CAP-treated lymphocytes are incubated for 24 hours (304). The T lymphocyte exosomes are then harvested using an isolation method (402). For example, the conditioned media can be collected and differentially centrifuged at 300×g, 1500×g, 4500×g and 10,000×g. The supernatant collected and the final centrifuged step are mixed with exosome isolation solution (Total Exosome Isolation Reagent, Thermo) and incubated overnight at 4 C. The next day the conditioned media is centrifuged at 10,000×g and the exosome pellets are collected and resuspended in appropriate vol of PBS and stored at −80 C until used. The application of CAP to the process of isolating and harvesting exosomes dramatically increased the exosome production by as much as seven times.

### Example

A more specific method in accordance with the present invention to isolate and harvest T lymphocyte exosomes has four phases: (1) isolation of T Lymphocytes; (2) culture of T Lymphocytes; (3) CAP treatment for secretion of exosomes from Human T Lymphocytes; and (4) harvesting exosomes from Human T Lymphocytes.

The T Lymphocytes are isolated through the following steps:
Obtain human blood from a healthy donor. Allow the blood to cool to room temperature (˜30 min) before proceeding to the next step.
Gently pipette 3 mL of room temperature Polymorph density gradient media into an 8 mL round-bottom polystyrene tube. Gently add 3 mL of whole blood on top of the Polymorph media. It is important to avoid mixing of the two reagents.
Centrifuge the tubes at 500×g for 45 minutes at room temperature.
Following the centrifugation, the peripheral blood mononuclear cells (PBMC) have now separated from other blood components into the top cell layer. The PBMC layer appears, from the top down, as the first cloudy band.
Carefully remove the clear yellow-colored upper phase of the blood, above the PBMC layer, and then use a P1000 micropipette to transfer the PBMC layer to a 15 mL or 50 mL conical tube.
Wash the PBMC twice with PBS, centrifuging cells at 500×g for 5 minutes each time. The supernatant will be somewhat cloudy after each wash.

The T Lymphocytes are cultured through the following steps:
Using a pipette, transfer the PBMC to a T-75 culture flask in 20 mL RPMI 1640 media containing 10% FBS, 1% penicillin/streptomycin, and 1 μg/mL phytohemagglutinin (PHA).
Incubate at 37° C. and 5% CO₂ for at least 1 hour, and up to 24 hours. This step allows monocytes, which will be adherent to the flask surface, to be separated from the lymphocytes that remain in suspension. If a short incubation (1 hour) is used at this step, it is acceptable to use RPMI 1640 media containing 10% FBS and 1% penicillin/streptomycin without supplementing with PHA as specified in step 2.1.
Carefully remove all of the media from the flask, add it to a 50 mL conical tube, and centrifuge at 500×g for 5 minutes.
Resuspend the cell pellet, which now primarily contains lymphocytes, and transfer the cells to a new T-75 flask containing 25 mL RPMI 1640 media containing 10% FBS, 1% penicillin/streptomycin, and 1 μg/mL PHA.
Incubate at 37° C. for 3 days (2 days if the initial incubation of PBMC was overnight). After 24 hours of growth, it may be necessary to add 15-20 mL of fresh media and transfer to a larger T-175 flask.
After 3 days, use a pipette to remove the media and suspended lymphocytes from the flask and transfer to a 50 mL conical tube. Centrifuge at 500×g for 5 minutes.
Resuspend the cell pellet and transfer cells to a new T-75 or T-175 flask containing 25 mL (T-75) or 50 mL (T-175) RPMI 1640 with 10% FBS, 1% penicillin/streptomycin, and 20 ng/mL human IL-2 or IL-15.
Grow lymphocytes for 4-7 days. If starting with a T-75 flask, the culture will need to be expanded and transferred to a T-175 flask after 1-2 days.

CAP treatment for secretion of exosomes from Human T Lymphocytes is performed through the following steps:
Transfer 100K T lymphocytes to each well on a 12 wells culture plate in 1 mL RPMI 1640 media containing 10% FBS, 1% penicillin/streptomycin, and 20 ng/mL human IL-2 or IL-15.
Culture for 2 days and replace the media to RPMI 1640 media containing 10% NU serum, 1% penicillin/streptomycin, and 20 ng/mL human IL-2 or IL-15.
After hour incubation, carryout Canady Cold Atmospheric Plasma (CAP) treatment at 120p for 3 minutes at 3 L Helium supply.
Incubate the plates at 37° C. and 5% CO₂ for the required period.

Harvesting exosomes from Human T Lymphocytes is performed through the following steps:
1. After incubation, Collect the media and centrifuge at 300×g for 10 mins at 4 C, collect the supernatant.
Centrifuge the supernatant at 1500×g for 10 mins at 4 C, collect the supernatant.
Centrifuge the supernatant at 4500×g for 10 mins at 4 C, collect the supernatant
Transfer the required volume of cell-free culture media to a new tube and add 1:1 volume of JCRI Exosome Isolation reagent and mix well.
Mix the culture media/reagent mixture well by vortexing, or pipetting up and down until there is a homogenous solution
Incubate samples at 2° C. to 8° C. overnight.
After incubation, centrifuge the samples at 10,000×g for 1 hour at 2° C. to 8° C.
Carefully aspirate and discard the supernatant. Exosomes are contained in the pellet at the bottom of the tube (not visible in most cases).
Resuspend the pellet in a convenient volume of 1×PBS or culture media.
Keep isolated exosomes at 2° C. to 8° C. for up to 1 week, or at <80° C. for long-term storage.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for isolation and culturing of microvesicles, comprising:
isolating peripheral blood mononuclear cells from human blood;
culturing the isolated peripheral blood mononuclear cells;
treating the cultured peripheral blood mononuclear cells with cold atmospheric plasma for secretion of microvesicles; and
harvesting microvesicles from the CAP-treated peripheral blood mononuclear cells.

2. A method according to claim 1, wherein the harvested microvesicles comprise T lymphocyte exosomes.

3. A method according to claim 1, wherein the step of isolating peripheral blood mononuclear cells from human blood comprises:
adding human blood to a polymorph density medium in a first flask;
centrifuging the first flask of blood and polymorph density medium to separate the peripheral blood mononuclear cells from other blood components;
collecting the peripheral blood mononuclear cells from the first flask and transferring the peripheral blood mononuclear cells to a second flask;
centrifuging the second flask of peripheral blood mononuclear cells; and
washing the peripheral blood mononuclear cells in the second flask.

4. A method according to claim 3, wherein the step of culturing the peripheral blood mononuclear cells comprises:
culturing the washed peripheral blood mononuclear cells in selective media;
removing the peripheral blood mononuclear cells from the selective media and transferring them to a third flask;
centrifuging the peripheral blood mononuclear cells in the third flask;
transferring the centrifuged peripheral blood mononuclear cells from the third flask to a fourth flask;
centrifuging the peripheral blood mononuclear cells in the fourth flask;
culturing the centrifuged peripheral blood mononuclear cells in the fourth flask in selective media containing IL-2 or IL-15; and
replacing the selective media with exosome free fetal bovine serum.

5. A method according to claim 4, wherein the step of harvesting microvesicles from the CAP-treated peripheral blood mononuclear cells comprises an isolation method.

6. A method according to claim 5, wherein the isolation method comprises:
incubating the CAP-treated peripheral blood mononuclear cells;
differentially centrifuging the incubated CAP-treated peripheral blood mononuclear cells;
mixing the centrifuged incubated CAP-treated peripheral blood mononuclear cells with an exosome isolation solution;
incubating the exosome isolation solution and centrifuged incubated CAP-treated peripheral blood mononuclear cells;
centrifuging the incubated exosome isolation solution and centrifuged incubated CAP-treated peripheral blood mononuclear cells; and
collecting microvesicle pellets from the centrifuged incubated exosome isolation solution and centrifuged incubated CAP-treated peripheral blood mononuclear cells.
